(19) United States
(12) Patent Application Publication
(10) Pub. No.: US 2004/0172878 A1
(43) Pub. Date: Sep. 9, 2004

(54) Method and system of generating methane and electrical energy and thermal

(76) Inventors: Adam Krylowicz, Zamosc (PL); Kazimierz Chrzanowski, Zamosc (PL); Janusz Usidus, Zamosc (PL)

Correspondence Address: BURNS DOANE SWECKER & MATHIS L L P, POST OFFICE BOX 1404, ALEXANDRIA, VA, 22313-1404, US

(21) Appl. No.: 10/483,298

(22) PCT Filed: Jul. 3, 2002
(86) PCT No.: PCT/PL02/00044

(30) Foreign Application Priority Data
Jul. 12, 2001 (PL) P.348681

(51) Int. Cl. C01B 3/32; C12P 5/00

(52) U.S. Cl. 48/127.3; 48/127.9; 435/166; 435/290.4; 435/300.1

(57) ABSTRACT

The method according to the invention concerns an anaerobic conversion of biomass into biogas in separated processes of hydrolysis and methane fermentation of biomass by means of methane mesophile, thermophile and psychrophile bacteria, contained in returned reflux. Cleaned biogas undergoes decomposition into methane and carbon dioxide. From part of methane and biogas standard gas fuel is produced, used for the engine of a current generating unit and a thermoregenerative cell generating electrical energy and heat. The system according to the invention consists of a system of preparation of biomass (1) connected to a hydrolyser (2) and then to a series system of fermentation tanks and a composter (1), which co-operates with a system of returning and enriching reflux (4). A tank for raw biogas (5) is connected to a system for cleaning biogas (6) and then to a tank for cleaned biogas (7) connected to a system of biogas decomposition (8) and a gas mixer (11). The system (8) has outlets to the system of CO₂ processing (10) and to the system of methane processing (2) also connected to the gas mixer (11) connected to a tank for standard gas fuel (12), The tank (12) has a connection to the system of generating electrical energy and heat (13) and the system of heat processing (14).

The subject of the invention is the method of generating methane and electrical energy and thermal, especially from plants grown specifically for this purpose.

According to Witold M. Lewandowski's “Pro-ecological sources of renewable energy”, WNT, Warszawa 2001, there are three main sources of biogas:

1) fermentation of active deposit in fermentation tanks of sewage treatment plants,

2) fermentation of organic industrial and consumption waste in waste dumps,

3) fermentation of manure and liquid manure in individual agricultural farms.

The book mentioned above also describes ways of production and utilisation of biogas from these sources.

W. Romaniuk in his book entitled: “Ecological systems of manure and liquid manure management”, IBMER, Warszawa 2000, describes the method and system of utilising manure according to “eurotechnology” developed by the Institute of Agriculture Construction, Mechanisation and Electrification. Utlisation of manure, according to “eurotechnology” is based on warming of manure in heat exchangers to the temperature of 35° C., forcing the warmed-up manure to the fermentation tank in such a way that the amount of fermented manure which leaves the fermentation tank and flows to the manure chambers is the same as the amount of the fresh manure which was initially forced into the chamber. Manure introduced to the fermentation tank undergoes anaerobic conversion of biomass into biogas by means of methane mesophile bacteria over a period of over 20 days and is stirred energetically three times a day. Biogas received as a result is burnt in a burner or is used as gas fuel for gas engines of water-cooled current-generating units. Part of regained heat is used to warm up fresh manure introduced to the fermentation tank.

The system of manure utilisation is composed of an introductory tank for manure, heat exchangers: manure/manure and water/manure, a fermentation tank, a biogas desulphuriser, a tank for biogas, a water-cooled current-generating 380 V unit, and manure chambers. Similar systems are used in utilisation of manure together with plant waste and other organic waste.

From patent no. P-318982, entitled: “The way of generating energy and the thermoregenerative cell”—we know the method of generating electrical energy of direct current by means of the synthesis of hydrogen with halogen in a thermoregenerative cell, e.g. with iodide to hydrogen iodide dissolving in electrolyte—hydrogen iodide acid—causing the increase of concentration of hydrogen iodide acid; then hydrogen iodide is expelled from the concentrated acid in a low-temperature thermoregenerator, preferably at a temperatureof 100° C.; then iodide hydrogen undergoes a thermal decomposition in a high-temperature thermoregenerator into iodide and hydrogen, preferably at a temperatureof 400° C. Following the physical decomposition into hydrogen and iodide, hydrogen is returned to the hydrogen electrode and iodide to the iodide electrode in the cell.

The method and system of generating biogas and electrical and thermal energy from sewage deposits from sewage treatment plants is known from J. Gańczarczyk's book entitled: “Water supply systems and sewage systems, Manual”, Arkady, Warszawa 1971.

Utilisation of sewage deposits is made by forcing sedimentary sewage solids containing about 4% of dry mass in water into heat exchangers, where it is heated until it reaches the temperature of about 25,5° C.; then it is forced into fermentation tanks, where there is a stable temperature of about 23° C.; then the deposits undergo methane fermentation by methane psychrophile bacteria.

The liquid with the deposits is stirred and the deposits remain in fermentatitanks for about 20 days. Biogas received in this way undergoes desulphurisation and is burnt in combustion engines of current-generating units, whereas generated electrical energy is transferred to an electrical network, usually to be used in a sewage treatment plant; the surplus of biogas is burnt in a gas torch. Some of the heat from the combustion gases is recovered in heat exchangers and is used for heating the deposits directed to the fermentation tanks. According to this patent proposition the deposit utilisation system is composed of a deposit decanter, deposit pumps, heaters, fermentation tanks, a biogas desulphuriser, a biogas tank, current-generating units, a gas torch, a press dehydrating fermented deposits, and a mixer for dehydrated deposits and burnt lime. Biogas received in these ways is characterised by variable contents of methane, and so a variable methane number and variable heat value, which has a bad influence on the work of combustion engines of current-generating units and lowers their efficiency and life. Methane fermentation of biomass by means of methane psychrophile or mesophile bacteria is characterised by lower efficiency of producing methane from a unit of dry biomass mass compared to fermentation by means of methane thermophile bacteria. However, methane thermophile fermentation of biomass conducted at a temperatureof about 55° C. requires delivering more heat to fermentation tanks than is required for methane mesophile fermentation at a temperatureof about 35° C., or methane psychrophile fermentation at a temperatureof 23° C. Moreover, methane fermentation of manure or sewage deposits is characterised by low efficiency of producing methane from a unit of dry mass—usually less than 300 m³ of methane from the ton of dry mass of such biomass. At the same time there is less than 10% of dry mass in the solution. What is more, methane fermentation takes longer—over 20 days—in order to destroy parasite eggs, pathogenic bacteria, and to decrease the disagreeable odour of manure or sewage deposits. All this contributes to the fact that building such big fermentation tanks is very expensive and it is difficult to control the methane fermentation processes of such biomass.

The invention solves the problem of using specially grown plants and organic waste and complete utilisation of biomass to produce methane, electrical and thermal energy and compost. It also solves the problem of controlling the process of anaerobic conversion of biomass into biogas and effective conversion (over 60%) of chemical energy of the received fuel into electrical energy.

All this results from separating the processes of biomass hydration, mesophile, thermophile and psychrophile methane fermentation and composting used biomass in each of these technological processes by means of returning the reflux containing suitable bacterial cultures to wet the biomass introduced in these processes, and also by decomposition of clean biogas obtained by these processes into methane and carbon dioxide and producing standard gas fuel, and also by associating generating electrical energy by a current-generating unit or a current-generating turbo set and a thermoregenerative cell, and by complete utilisation of produced heat for technological processes.

Generating methane and electrical and thermal energy by means of anaerobic conversion of biomass in the form of crushed plants grown specially for this purpose and/or organic waste into biogas, and employing a thermoregenerative cell and a current-generating unit or a current-generating turbo set to produce electrical and thermal energy, is characterised by the fact that crushed plants are mixed with water in such a way that the contents of dry mass in water is 20% to 60%, preferably 30%. In the same proportion, crushed organic waste is mixed with water. At the beginning it contains 60% of water. These mixtures, together with organic waste containing from 4% to 20% of dry mass in water, undergo together, separately, or in specific sets, hydrolysis at a temperatureof about 20° C. over the period of 12-36 hours. Then, carbon dioxide is forced through this hydrolysed biomass until a complete disappearance of oxygen and nitrogen in the biomass. Then, if necessary, water is added to the mixture until the amount of dry mass is from 4% to 60%, preferably 20%, and biomass undergoes methane fermentation by means of methane mesophile bacteria, preferably at a temperatureof 35° C. over the period of 48-240 hours. Biogas produced in the anaerobic process of converting biomass into biogas—further on called the first portion—is directed to the tank for raw biogas, and the remaining biomass is, if necessary, refilled with water, until it contains from 4% to 60%, preferably 20% of dry mass, and it undergoes methane fermentation by means of methane thermophile bacteria, preferably at a temperatureof 55° C. over the period of 48-240 hours. In both processes of methane fermentation, the proportion of carbon to nitrogen in biomass is over 100:3, it is preferably 10:1, at the pH of the water biomass mixture from 6 to 8—preferably pH=7, and its redox potential lower than 250 mV. Biogas produced in the anaerobic process of converting biomass into biogas by means of methane thermophile bacteria—further on called the second portion—is combined with the first portion in the tank for raw biogas, and the rest of biomass, after extracting of about 50% of water from it and returning the water to the methane fermentation process of the next portion of biomass, is composted, preferably at a temperatureof 23° C. over the period of 190-300 hours, with the process of anaerobic converting of biomass into biogas by means of methane psychrophile bacteria going on simultaneously. Then the resulting compost is used in agriculture as natural fertiliser. The biogas produced, which constitutes the third portion, is combined with the previous biogas portions; sulphur compounds are removed from them, and then 20%-80% of this desulphurised biogas is decomposed into methane and carbon dioxide, of which 5% to 50% accumulates in a tank under higher pressure, and which then is again returned to the process of removing oxygen and nitrogen from the hydrolysed biomass. The rest of the carbon dioxide is accumulated in gas bottles under higher pressure, or is condensed, or is expelled to the atmosphere. 25%-75% of methane is either condensed, combined with natural gas, used in its clean form as fuel, or is converted into other chemical compounds, whereas the rest of the methane, or 100% of the methane produced, is combined with the portion of desulphurised biogas which did not undergo decomposition, in the proportion necessary to get gas fuel of a constant methane number, preferably 104,4 and a constant heat value of about 8,6 kW/m³—called standard gas fuel. 20%-40% of this fuel is burnt in a thermoregenerator burner of a high-temperature thermoregenerative cell causing thermal decomposition of the synthesis products accumulated in the cell and regeneration of the reducer and oxidiser The latter are returned to the electrodes of the cell, which results in the generating of electrical energy of direct current in the cell. Additionally there is an increase of concentration of electrolyte directed from the cell to the low temperature thermoregenerator, whereas the rest of the fuel is burnt in the combustion engine of a current-generating unit generating electrical energy of variable current and heat contained in the liquids cooling the engine and in combustion gases, or is burnt in the combustion chamber of a current-generating turbo set generating electrical energy of variable current and heat contained in the combustion gases emitted from a gas turbine. 25%-75% of the heat recovered from the engine cooling liquids and from combustion gases, is delivered to the low temperature thermoregenerator of the thermoregenerative cell to take part in the process of emitting the synthesis products from electrolyte and returning them to the thermoregenerator of the high-temperature cell and returning the low concentrated electrolyte to the chambers of the cell, whereas 25%-75% of heat is delivered to the processes of hydrolysis and anaerobic conversion of biomass into biogas. The remaining heat is delivered to a central heating system and/or is used to produce warm water. Reflux formed in a particular technological cycle is returned to be reused in this cycle. Reflux directed to the fermentation tanks is completed, in particular, nitrogen compounds are added.

Moreover, the subject matter of the invention is the system of generating methane and electrical and thermal energy.

The system of generating methane and electrical and thermal energy, composed of a hydrolyser, fermentation tanks, an expeller, a composter, a current-generating unit or a current-generating turbo set, a thermoregenerative cell, tanks, gas and liquid pumps and pipelines, a system of biomass preparation connected to the hydrolyser, which in turn is connected to a series system of fermentation tanks and a composter, which is equipped with a compost conveyor to a storage site and a net of connections with a system of returning and enriching reflux. These systems: the system of biomass preparation, the series system of fermentation tanks and a composter, and the system of returning and enriching reflux are connected to an outer water intake, whereas the series system of the fermentation tanks and a composter is connected to a tank for raw biogas. This tank is connected to a system of biogas cleaning, which in turn is connected to a tank for cleaned biogas. The tank for cleaned biogas is connected to a system of biogas decomposition and to a gas mixer. The system of biogas decomposition is connected to a system of carbon dioxide processing and a system of methane processing. The system of carbon dioxide processing is connected by means of a gas pipeline to the hydrolyser, and it is also equipped with an outlet of carbon dioxide to the atmosphere. The system of methane processing is connected to a gas mixer, which in turn is connected to a tank for standard gas fuel. This tank is linked to a system of electrical energy and heat generating and alternatively is connected to a system of heat processing. The system of electrical energy and heat generating is connected to the system of heat processing, which in turn is connected by means of heat pipelines to the hydrolyser, the system of reflux returning and enriching and the series system of fermentation tanks and a composter. The system of biomass preparation is composed of a biomass mixer connected to the hydrolyser and the outer water intake by means of a water pipeline of the biomass mixer. It is also connected to a grass, cereal, and leaf cutter, to a root plants cutter and also to a storage site or a tank for organic waste, especially if the organic waste has the form of sedimentary solids in water. The hydrolyser linked at the entry to the biomass mixer and at the outlet to a conveyor for hydrolysed biomass, contains a secondary water cycle of the hydrolyser, coming out at the bottom of the hydrolyser from under the conveyor for hydrolysed biomass and getting in at the top of the hydrolyser near the entry to the hydrolyser of biomass prepared by the system of biomass preparation. At the bottom there is also a feeder of CO₂ to the hydrolyser, and at the top there is an outlet of gases from the hydrolyser, there is also a water heater of the heating system of the hydrolyser and fermentation tanks. The series system of fermentation tanks and a composter is composed of a mesophile fermentation tank, a thermophile fermentation tank, an expeller and a composter, linked in series by means of a biomass conveyor; at the same time the mesophile fermentation tank has, at the entry, a conveyor for hydrolysed biomass, and at the outlet a conveyor for biomass after mesophile fermentation. This conveyor is linked to the thermophile fermentation tank, which at the outlet has a conveyor for biomass after thermophile fermentation connected to the expeller. The expeller is in turn connected by means of a conveyor for pressed biomass to a composter, which is equipped with a leakproof gas chamber, and at the outlet, a conveyor of compost to the storage site Both fermentation tanks are equipped with water heaters from the heating system of the hydrolyser and fermentation tanks. The gas chambers of the fermentation tanks and the composter are connected by means of gas pipelines to the tank for raw biogas, connected by means of a pipeline for raw biogas to the system for cleaning biogas. The system of returning and enriching of reflux is composed of the secondary water cycle of the mesophile fermentation tank coming out at the bottom of the mesophile fermentation tank from under the conveyor for biomass after mesophile fermentation and getting into the fermentation tank at the top near the entry to the fermentation tank of the conveyor for hydrolysed biomass, of the secondary water cycle of the thermophile fermentation tank. getting out at the bottom of the thermophile fermentation tank from under the conveyor for biomass after mesophile fermentation, and getting into the fermentation tank at the top near the entry into the fermentation tank of the conveyor for biomass after mesophile fermentation. It is also composed of the secondary water intake of the expeller connected to the secondary water cycle of the thermophile fermentation tank, and also of the secondary water cycle of the composter, getting out at the bottom of the composter and getting into the composter at the top near the entry to the composter of the conveyor for pressed biomass. Both these cycles are connected to the outer water intake by means of an outer water pipeline. The secondary water cycles of the mesophile and thermophile fermentation tanks are joined to a feeder of nitrogen compounds. The system of biogas decomposition consists of a two-chambered saturator and a liquid cycle of the saturator. The entry chamber A of the saturator is filled with liquid absorbing only carbon dioxide from a gas mixture, and is equipped at the outlet with a gas pipeline for methane. Inside the saturator chamber A is linked to the exit chamber B of the saturator, filled with the same liquid emitting CO₂. At the top it is connected with a gas pipeline for CO₂ and at the bottom with a pipeline for liquid of the liquid cycle of the saturator, getting into chamber A, and used for returning liquid from chamber B to chamber A Chamber A of the saturator is connected by means of a gas pipeline below the liquid level in the chamber with the tank for cleaned biogas, and then with the system of raw biogas cleaning consisting of a column for biogas desulphurisation and a gas pump. The system of carbon dioxide processing is composed of a gas pipeline for carbon dioxide joining the saturator and the CO₂ feeder to the hydrolyser. Moreover, a tank for compressed carbon dioxide and a condensing CO₂ unit are connected to the pipeline.

The condensing CO₂ unit is connected from the other side to the tank for condensed carbon dioxide. This pipeline also has a controlled outlet of carbon dioxide to the atmosphere. The system of methane processing is composed of a gas pipeline for methane, getting out of the saturator and connected to a methane condensing unit, which is further on connected to a tank for condensed methane, or connected to a gas main, also connected to a gas mixer, which is linked at the entrance to the tank for cleaned biogas, and at the exit to the tank for standard gas fuel. The system of generating electrical energy and heat is composed of a current-generating unit, which has an electrical connection with a power network, and a theromoregenerative cell which is equipped with a high-temperature thermoregenerator and a low-temprature thermoregenerator. The combustion engine of the current-generating set and the high-temperature thermoregenerator of the cell are connected by means of a pipeline for standard gas fuel to the tank for standard gas fuel, and the pipeline has an anti-failure connection with a gas torch The low-temperature thermoregenerator of the cell is also equipped with a heat exchanger connected to a heat combustion gases/liquid exchanger in the system of heat processing. The system of heat processing is composed of a main heat cycle, the heating system of the hydrolyser, and fermentation tanks, the heat cycle of central heating, and the heat cycle of the low-temperature thermoregenerator. In the main heat cycle there is a water pump of the heat cycle connected to a heat exchanger liquid/liquid in the cycle of liquids cooling the engine, and then to a heat exchanger combustion gases/liquid absorbing heat from combustion gases. Then, the main heat cycle is connected to the heat cycle of central heating and the heating system of the hydrolyser and fermentation tanks, equipped with water heaters situated in the hydrolyser and in the fermentation tanks. The heat cycle of the low-temperature thermoregenerator connects the heat exchanger combustion gases/liquid to the heat exchanger of the low-temperature thermoregenerator. In an alternative system of generating electrical energy and heat, a gas turbine has been installed, which is connected at the synchro-tie to a three-faze current generator in place of the current-generating unit. The pipeline for standard gas fuel is connected to the combustion chamber of a gas turbine, and the combustion outlet of the gas turbine is connected to a heat exchanger heating compressed air which is forced through to a combustion chamber of gas fuel, and then in turn to a heat exchanger combustion gases/liquid in the main heat cycle of the system. The three-faze current generator is connected electrically to a power network

The subject of the invention is illustrated by drawings.

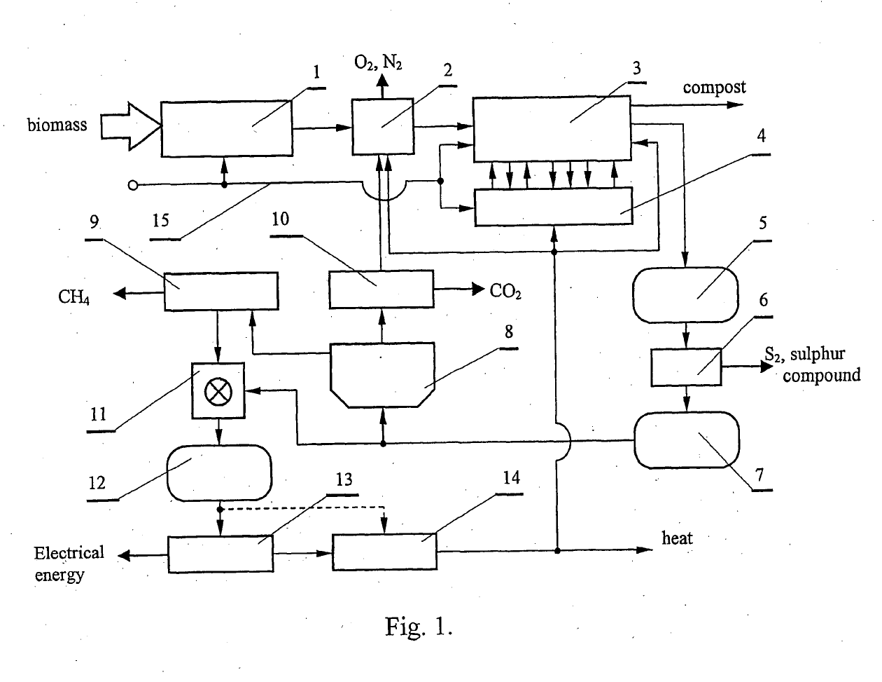

FIG. 1 shows a diagram of the technological process which illustrates how the systems taking part in the technological process of generating methane and electrical and thermal energy are connected.

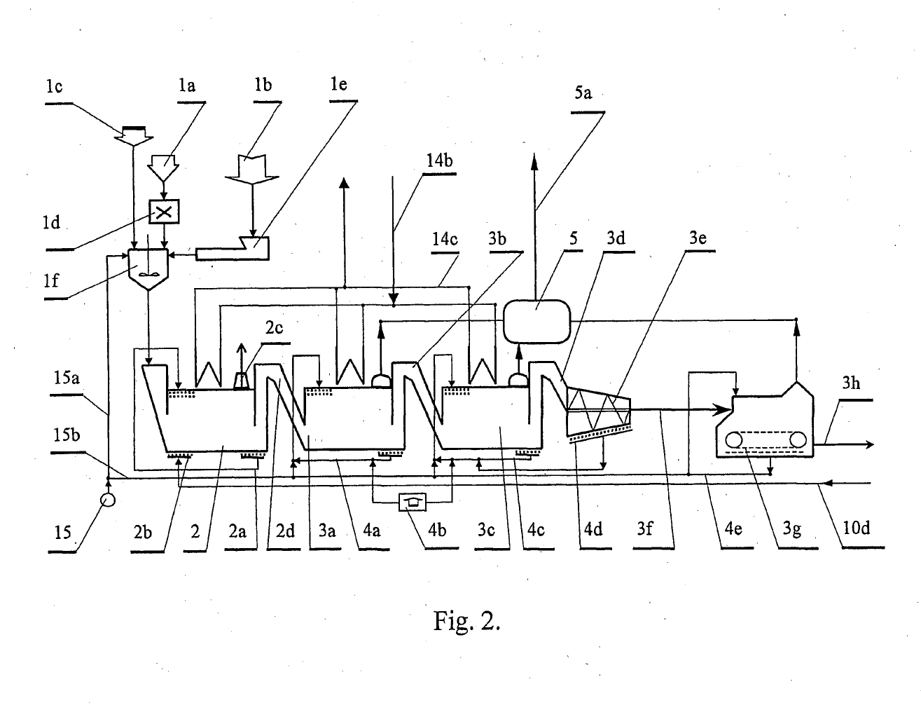

FIG. 2 illustrates the system of preparation biomass, the hydrolyser, the series system of fermentation tanks and a composter, the tank for raw biogas, the outer water intake and the system of returning and enriching reflux.

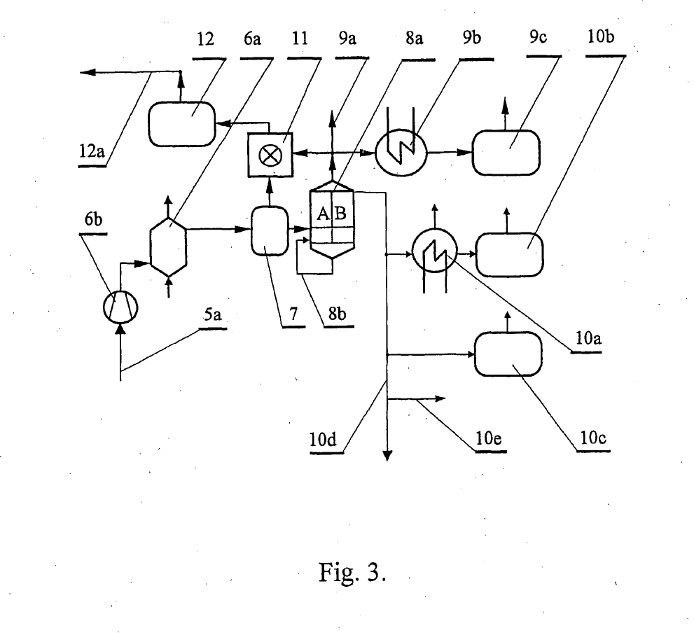

FIG. 3 illustrates the system for cleaning biogas, the system of biogas decomposition, the system of carbon dioxide processing, the system of methane processing, and the gas mixer and technological tanks.

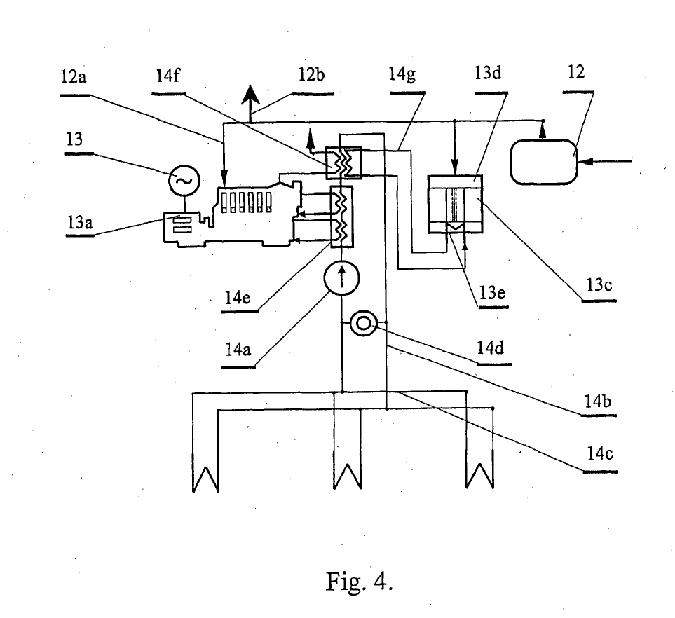

FIG. 4 illustrates the system of electrical energy and heat generating and the system of heat processing.

FIG. 1 shows a diagram of the technological process of generating methane, electrical energy and thermal energy, which consists of a system of biomass preparation 1, a hydrolyser 2, a series system of fermentation tanks and a composter 3, a system of returning and enriching reflux 4, a tank for raw biogas 5, a system for cleaning biogas 6, a tank for cleaned biogas 7, a system of biogas decomposition 8, a system of methane processing 9, a system of carbon dioxide processing 10, a gas mixer 11, a tank for standard gas fuel 12, a system of electrical energy and heat processing 13, a system of heat processing 14 and the outer water intake 15. The system of biomass preparation is connected to the hydrolyser 2, which in turn is connected to the series system of fermentation tanks and a composter 3 which is equipped with a conveyor of compost to the storage site and is connected to the system of returning and enriching reflux. These systems: the system of biomass preparation, the series system of fermentation tanks and a composter, and the system of returning and enriching reflux are connected to the outer water intake 15. The series system of fermentation tanks and a composter 3 is connected to the tank for raw biogas 5. This tank is connected to the system for cleaning biogas 6, which in turn is connected to the tank for cleaned biogas 7. The tank for cleaned biogas is connected to the system of biogas decomposition 8 and the gas mixer 11. The system of biogas decomposition is connected to the system of carbon dioxide processing 10 and the system of methane processing 9. The system of carbon dioxide processing is connected by means of a gas pipeline to the hydrolyser 2 and it is also equipped with an outlet of CO₂ to the atmosphere. The system of methane processing 9 is also connected to the gas mixer 11, which in turn is connected to the tank for standard gas fuel 12. This tank has a connection with the system of generating electrical energy and heat 13 and an alternative connection to the system of heat processing 14. The system of electrical energy and heat generating 13 is connected to the system of heat processing 14, which in turn is connected by means of a heat pipeline to the hydrolyzer 2, to the system of returning and enriching reflux 4 and the series system of fermentation tanks and a composter 3.

FIG. 2 illustrates a system of biomass preparation, a hydrolyser, a series system of fermentation tanks, and a composter, a tank for raw biogas and a system of reflux returning and enriching. The system of biomass preparation consists of a biomass mixer 1f connected to the hydrolyser 2 and to an outer water intake 15 by means of a water pipeline of the biomass mixer 5a, it is also connected to a grass, leaves and cereal plant cutter 1a and to a cutter 1e of root plants 1b and it is also connected to a storage site or a tank for organic waste 1c, especially when it is in the form of sedimentary solids in water. The hydrolyser is at the entrance connected to the biomass mixer 1f and at the exit it is equipped with a conveyor for hydrolysed biomass 2d, it also has a secondary water cycle of the hydrolyser 2a coming out at the bottom of the hydrolyser from under the conveyor for hydrolysed biomass and getting in at the top of the hydrolyser near the entrance to the hydrolyser of biomass prepared by the system of biomass prepration. It is also equipped with a feeder of CO₂ to the hydrolyser 2b, and at the top there is an outlet 2c of gases from the hydrolyser; there is also a water heater of the heating system of the hydrolyser and fermentation tanks 14c connected by means of a heat pipeline 14b to the main heat cycle. The series system of fermentation tanks and a composter is composed of a mesophile fermentation tank 3a, thermophile fermentation tank 3c, an expeller 3e and a composter 3g, linked in series by means of biomass conveyors, at the same time the mesophile fermentation tank has at the entry a conveyor for hydrolysed biomass 2d, and at the outlet a conveyor for biomass after mesophile fermentation 3b. This conveyor is linked to the thermophile fermentation tank 3c, which at the outlet has a conveyor for biomass after thermophile fermentation 3d connected to an expeller 3e. The expeller is in turn connected by means of a conveyor for pressed biomass 3f with a composter 3g, which is equipped with a leakproof gas chamber, and at the outlet, a conveyor of compost to the storage site 3h. Both fermentation tanks are equipped with water heaters of the heating system of the hydrolyser and fermentation tanks 14c. The gas chambers of the fermentation tanks and a composter are connected by means of gas pipelines to the tank for raw biogas 5, connected by means of a pipeline for raw biogas 5a to the system for cleaning biogas. The system of returning and enriching reflux is composed of the secondary water cycle of the mesophile fermentation tank 4a coming out at the bottom of the mesophile fermentation tank 3a from under the conveyor for biomass after mesophile fermentation 3b and getting into the fermentation tank at the top near the entry to the fermentation tank of the conveyor for hydrolysed biomass 2d, of the secondary water cycle of the thermophile fermentation tank 4c and getting out at the bottom of the thermophile fermentation tank 3c from under the conveyor for biomass after mesophile fermentation 3d, and getting into the fermentation tank at the top near the entry into the fermentation tank of the conveyor for biomass after mesophile fermentation 3b. It is also composed of the secondary water intake of the expeller 4d connected to the secondary water cycle of the thermophile fermentation tank 4c, and also of the secondary water cycle of the composter 4e getting out at the bottom of the composter and getting into the composter at the top near the entry to the composter of the conveyor for pressed biomass 3f. Both these cycles are connected to the outer water intake 15 by means of an outer water pipeline 15b. The secondary water cycles of the mesophile and theromophile fermentation tanks are joined to a feeder of nitrogen compounds 4b.

FIG. 3 illustrates a system for cleaning biogas, a system of biogas decomposition, a system of carbon dioxide processing, a system of methane processing, a gas mixer and technological tanks. The system for cleaning biogas consists of a column for biogas desulphurisation 6a connected at the entrance to a gas pump 6b, and at the exit to a tank for cleaned biogas 7. The gas pump 6b is connected to the tank for raw biogas by means of a raw biogas pipeline 5a. The system of biogas decomposition consists of a two-chambered saturator 8a and a liquid cycle of the saturator 8b. The entry chamber A of the saturator is filled with liquid absorbing only carbon dioxide from a gas mixture, and is equipped at the outlet with a gas pipeline for methane 9a. Inside the saturator, chamber A is linked to the exit chamber B of the saturator, filled with the same liquid emitting CO₂. At the top it is connected to a gas pipeline for CO₂ 10d and at the bottom to a pipeline for liquid of the liquid cycle of the saturator 8b, getting into chamber A and used for returning liquid from chamber B to chamber A. Chamber A of the saturator is connected by means of a gas pipeline below the liquid level in the chamber to the tank for cleaned biogas 7. The system of carbon dioxide processing is composed of a gas pipeline for carbon dioxide 10d connecting the saturator 8a and the CO₂ feeder to the hydrolyser. Moreover, a tank for compressed carbon dioxide 10c and a condensing CO₂ unit 10a are connected to the pipeline. The condensing CO₂ unit is connected from the other side to the tank for condensed carbon dioxide 10b. This pipeline also has a controlled outlet of carbon dioxide to the atmosphere 10e. The system of methane processing is composed of a gas pipeline for methane 9a, getting out of the saturator 8a and connected to a methane condensing unit 9b, which is further on connected to a tank for condensed methane 9c or connected to a gas main, also connected to a gas mixer 11, which is linked at the entrance to the tank for cleaned biogas 7, and at the exit to the tank for standard gas fuel 12

FIG. 4 illustrates the system of generating electrical energy and heat, and the system of heat processing. The system of generating electrical energy and heat is composed of a current-generating unit 13a, which has an electrical connection with a power network 13b, and a theromoregenerative cell 13c which is equipped with a high-temperature thermoregenerator 13d and a low-temprature thermoregenerator 13e. The combustion engine of the current-generating set, and the high-temperature thermoregenerator of the cell, are connected by means of a pipeline for standard gas fuel 12a to the tank for standard gas fuel 12, and the pipeline has an anti-failure connection with a gas torch 12b. The low-temperature thermoregenerator 13e of the cell is also equipped with a heat exchanger connected to a heat exchanger combustion gases/liquid 14f in the system of heat processing. The system of heat processing is composed of the main heat cycle, the heating system of the hydrolyser and fermentation tanks 14c, the heat cycle of central heating 14d and the heat cycle of the low-temperature thermoregenerator 14g. In the main heat cycle there is a water pump of the heat cycle 14a connected to a heat exchanger liquid/liquid 14e in the cycle of liquids cooling the engine, and then to the heat exchanger combustion gases/liquid 14f absorbing heat from the combustion gases. Then the main heat cycle by means of a heat pipeline 14b to the heat cycle of central heating 14d and the heating system of the hydrolyser and fermentation tanks 14c, equipped with water heaters situated in the hydrolyser and in fermentation tanks. The heat cycle of the low-temperature thermoregenerator 14g connects the heat exchanger combustion gases/liquid 14f to the heat exchanger of the low-temperature thermoregenerator 13e.

One of the advantages of the method of generating methane and electrical and thermal energy is generating methane and association of electrical and thermal energy and high efficiency (over 85% from specially grown plants and organic waste) which results in a closed CO₂ cycle in the atmosphere. The choice of plants contributes to high productivity of methane from a unit of dry mass of such biomass, which reaches even 840 m³/t. Moreover, the amount of dry mass in the solution in the fermentation tanks is greater than 20%, which contributes to the reduction of the size of fermentation tanks, calculated on a unit of biogas production in proportion to the size of fermentation tanks in the well-known systems of waste utilisation. The separation of the functions of the hydrolyser, mesophile fermentation tank, thermophile fermentation tank, and composter allows for returning to these devices of reflux containing suitable bacterial cultures, following the process of biomass processing, which makes it easier to control the anaerobic processes of biomass conversion into biogas and it also speeds up the processes. Whereas only part of biomass which was introduced to the hydrolyser at the beginning of the process goes into the thermophile fermentation tank at the highest temperature of 55° C., which contributes to reducing of heat utilisation in the system at the maximum biogas production from a unit of dry mass of biomass, as opposed to the present systems of waste utilisation. Biogas produced from plants does not contain sulphur compounds or very small amounts of such compounds. Separation of methane from carbon dioxide in the saturator allows for proper management of these gases. Part of CO₂ is used for removing used air from the hydrolyser, especially of oxygen, which is poisonous for methane bacteria, whereas part of CO₂ after condensing or compressing is of market value. Production of gas methane and/or condensed methane and simultaneously generating electrical and thermal energy allows for controlling of the amount of produced fuel electrical energy and thermal energy, if necessary. Mixing of biogas, clean from sulphur compounds, with methane guarantees receiving of standard gas fuel of a constant high methane number and a constant high heat value, which has a good influence on the work of a heat engine and its effectiveness. Decomposition of waste heat produced in the cooling system of a current-generating unit or a gas turbine into heat for the hydrolyser and fermentation tanks, heat for central heating and heat for a low-temperature thermoregenerator of a thermoregenerative cell—heat for the process of thermal decomposition of the electrolyte—allows for optimal utilisation of heat, depending on the season. Whereas introducing of the thermoregenerative cell to the heat cycle of a current-generating unit, or in another version of the invention, to the heat cycle of a gas turbine, allows for the generating of high electrical efficiency of such a system, which exceeds 60%.

The invention will be additionally explained by giving examples of methane generating and producing electrical and thermal energy by the system of generation of methane and electrical and thermal energy.

### EXAMPLE 1

Cleaned mangel 1b and ensilage of grass 1a are used as biomass for anaerobic biogas generation. Mangel crushed in the cutter 1e and ensilage crushed in the grass cutter 1d into particles not longer than 3 cm are mixed in the mixer 1f with water delivered from the outer water intake 15. In the mixer, the biomass undergoes further disintegration until the proportion of water to dry mass is 2:1. The biomass prepared in this way goes into the hydrolyser 2, where it is warmed up to the temperature of 20° C. and it undergoes the process of hydrolysis. Water trickling at the bottom of the hydrolyser is returned by means of the secondary water cycle of the hydrolyser 2a to the top part of the hydrolyser, all the time wetting the biomass in the hydrolyser. After the process of biomass hydrolysis, which takes 24 hours, the remaining oxygen and nitrogen are removed from the biomass through the gas outlet of the hydrolyser 2c, they are expelled by carbon dioxide, which is brought to the hydrolyser by the CO₂ feeder 2b at the bottom of the hydrolyser. The hydrolysed biomass is transported by means of the conveyor for hydrolysed biomass 2d to the mesophile fermentation tank 3a and at the very entrance is wetted by water whose temperature is 35° C. and which contains methane mesophile bacteria from the reflux received at the bottom of the fermentation tank and transported by means of the secondary water cycle of the mesophile fermentation tank 4a. This water is completed by water warmed up to the temperature of 35° C. from the outer water intake 15 transported by means of an outer water pipeline 15b, and it is enriched by nitrogen compounds from the feeder of nitrogen compounds 4b. As a result, in the mesophile fermentation tank 3a the proportion of water to the amount of dry mass of biomass is 5:1, the proportion of carbon to the amount of nitrogen in the biomass is 10:1, pH of the water mixture of the biomass is 6,5÷7, the redox potential of the mixture is lower than 250 mV and the temperature of the mixture is 35° C. The fermented biomass is stirred energetically for 10 minutes three times in every 24 hours. The time of methane fermentation of the biomass in the mesophile fermentation tank is 96 hours and biogas created as a result contains 85% CH₄ and 15% CO₂—as the first portion it accumulates in the tank for raw biogas 5. After 96 hours of methane fermentation, the amount of dry mass in the biomass decreased by 25%, because part of carbon from the biomass found itself in biogas and after mesophile fermentation the biomass is transported by means of the conveyor for biomass after mesophile fermentation 3b to the thermophile fermentation tank 3c and the excess of water from the biomass containing mesophile bacteria trickles to the secondary water cycle of the mesophile fermentation tank. The thick biomass transported to the thermophile fermentation tank 3c is wetted by water delivered by the outer water pipeline 15b and is warmed up to the temperature of 55° C., and also by water received from the reflux at the bottom of the thermophile fermentation tank, which contains methane thermophile bacteria and which is enriched in nitrogen compounds from the feeder of nitrogen compounds 4b and transported to the top of the fermentation tank by means of the secondary water cycle of the thermophile fermentation tank 4c. As a result, in the thermophile fermentation tank 3c, the proportion of water to the amount of dry mass of the biomass is 5:1, the proportion of the amount of carbon to the amount of nitrogen in the biomass is 10:1, the pH of the water mixture of biomass is about 7, the redox potential of the mixture is lower than 250 mV, and the temperature of the mixture is 55° C. The fermenting biomass is stirred energetically for 10 minutes three times in every 24 hours. The time of methane fermentation of the biomass in the thermophile fermentation tank is 96 hours and biogas containing 80 of % CH₄ and 20% of CO₂—as the second portion, accumulates in the tank for raw biogas 5. After 96 hours of methane thermophile fermentation, the biomass is removed from the fermentation tank and transported by means of the conveyor for biomass after thermophile fermentation 3d to the expeller 3e and the water reflux from the pressed biomass containing methane thermophile bacteria accumulating in the secondary water intake of the pug mil 4d is connected with the reflux of secondary water of the thermophile fermentation tank flowing to the secondary water cycle of the thermophile fermentation tank 4c and which is used for wetting the biomass brought to the thermophile fermentation tank. The biomass partly dehydrated by the expeller 3e is transported by the conveyor for pressed biomass 3f to the composter 3g, where it undergoes the final methane fermentation process by means of methane psychrophile bacteria at a temperatureof 23° C., regained biogas accumulates in the leak-proof gas chamber of the composter, and is further on processed into compost brought out by the compost conveyor 3h from the composter into the compost storage site. Water reflux containing methane psychrophile bacteria is returned to the composter by the secondary water cycle of the composter 4e to be sprinkled on the next portions of biomass in the composter. Composting time is 288 hours. Biogas from the composter containing 70% of CH₄ and 30% of CO₂—as the third portion, accumulates in the tank for raw biogas 5. Biogas from the tank for raw biogas is transported by the pipeline for raw biogas 5a to the gas pump 6b which increases the pressure of biogas until it reaches 800 kPa and then it is cleaned in the desulphurising column 6a from 0,01 of admixture of hydrogen sulphide contained in biogas in the well known Claus' process. Desulphurised biogas accumulates in the tank for cleaned biogas 7, from where 60% of biogas flows into the saturator 8a and 40% to the gas mixer 11. In the saturator, biogas flows under the pressure of 800 kPa through the layer of water in chamber A of the saturator, as a result, carbon dioxide from biogas dissolves in cold water, and methane, which does not dissolve in water, flows from chamber A of the saturator to the gas pipeline for methane 9a. Water solution saturated by carbon dioxide flows to the low-pressure chamber B of the saturator, gas pressure is lowered to 100 kPa, and carbon dioxide is expelled from water and forced into the CO₂ gas pipeline 10d. Water containing small amounts of CO₂ is returned by means of the water cycle of the saturator 8b under the pressure of 800 kPa to the high-pressure chamber A of the saturator; in this way, the water cycle in the saturator is closed. In the CO₂ condensing unit 1a 53% of carbon dioxide is condensed and the condensed gas accumulates in the tank for condensed CO₂ 10b as a product having market value; 10% of CO₂ after compression accumulates in the tank for compressed carbon dioxide 10c, and 37% of CO₂ flows through the controlled CO₂ outlet 10e to the atmosphere. Compressed carbon dioxide is supplied from the tank 10c by means of the CO₂ gas pipeline 10d to the CO₂ feeder to the hydrolyser 2b, to remove air used in the process of biomass hydrolysis 73% of methane from the gas pipeline for methane 9a is directed to the methane condensing unit 9b and condensed methane accumulated in the tank for condensed methane 9c as a product of market value, and 27% of methane flows to the gas mixer 11. In the gas mixer, biogas taken from the tank for cleaned biogas 7 is enriched in methane, and as a result standard gas fuel is created, whose constant methane number is 104,4 and constant heat value is 8,6 kWh/m³ and which is accumulated in the tank for standard gas fuel 12. This fuel is burned in the combustion engine of the current-generating unit. 13a connected to the electrical generator of three-faze current delivered to a power network 13b. The fuel is also burnt in the gas burner of a high-temperature thermoregenerator 13d of a thermoregenerative cell 13c generating direct current. A well-known hydrogen-iodide cell was used as the thermoregenerative cell. Heat from the process of cooling oil and from the water cooler of the current-generating unit is returned to the main heat cycle in the heat exchanger of the type oil/water and water/water 14c. Heat from the process of cooling combustion gases is returned to the same heat cycle in the heat exchanger combustion gases/water 14f. From the same heat exchanger by means of a separate heat cycle of the low-temperature thermoregenerator 14g 65% of heat flows into the low-temperature thermoregenerator 13e of the thermoregenerative cell, in which this heat causes thermal decomposition of the condensed electrolyte flowing from the cell—condensed hydrogen iodide acid produced in the cell—emitting from the electrolyte part of the hydrogen iodide in the form of gas and lowering the concentration of acid returned to the chambers of the cell. Hydrogen iodide undergoes thermal decomposition into iodide and hydrogen in the high-temperature thermoregenerator 13d and then hydrogen is separated from iodide on the diaphragm in the well-known way. Iodide as oxidiser is directed to the iodide electrode of the cell, and hydrogen as reducer flows to the hydrogen iodide of the cell, where there is a synthesis of hydrogen iodide which increases the concentration of the electrolyte and electrical energy of direct current is generated. Direct current is converted into three-faze current in an inverter. By means of thermal association of the current-generating unit and the thermoregenerative cell the amphere-hour efficiency of the system is 62%. In the heat cycle of the system, water forced by the pump of the heat cycle 14a flows, and 35% of heat is transported by a hot waterjet from heat exchangers 14e and 14f by means of a heat pipeline 14b to the heating system of the hydrolyser and the fermentation tanks. In this way the same temperature is kept in the heat hydrolyser and in the fermentation tanks. In the heating seasons the heat flows also to a system of central heating 14d.

### EXAMPLE II

Liquid manure 1c from a tank for liquid manure, cereal straw and grass ensilage 1a are used as biomass for anaerobic production of biogas. Straw and ensilage crushed in the cutter 1d are mixed in the biomass mixer 1f with liquid manure and water delivered from the outer water intake 15, in such a way that the biomass undergoes further disintegration until the proportion of water to dry mass is 5:1. The biomass prepared in this way goes into the hydrolyser 2, where it is warmed up to the temperature of 20° C. and it undergoes the process of hydrolysis over the period of about 24 hours. After the process of hydrolysis the biomass undergoes a further process of anaerobic conversion into biogas and compost in the fermentation tanks and a composter in the way described in example I, but there are longer periods of methane fermentation: the methane mesophile and thermophile fermentations in the fermentation tanks take 240 hours, and after this time the proportion of water to the amount of dry mass in the biomass in both fermentation tanks is 10:1. Similarly, the time of methane fermentation and composting of the biomass in the composter takes 240 hours Other parameters of the solutions are the same as in example I. The biogas created in the mesophile fermentation tank contains 70% of CH₄ and 30% of CO₂—as the first portion of biogas, the biogas created in the thermophile fermentation tank contains 65% of CH₄ and 35% of CO₂—it is the second portion of biogas, and the biogas created in the composter contains 60% of CH₄ and 40% of CO₂—it is the third portion of biogas; this last portion of biogas contains an admixture of 0,5% of _{H2S}. All these portions of biogas are put together in the tank for raw biogas 5 from where raw biogas flows by means of the pipeline for raw biogas 5a to the gas pump 6b which pumps biogas under a pressure of 150 kPa to the column of biogas desulphurisation. There, hydrogen sulphide from the biogas is combined with iron compounds in the bog iron ore and the cleaned biogas accumulates in the tank for cleaned biogas 7, from where 80% of the biogas flows into the low-temperature chamber A of the saturator 8a and 20% to the gas mixer 11. In chamber A of the saturator, filled with liquid monoethyloamine (MEA), carbon dioxide from biogas is combined with monomethyloamine creating under the pressure of 150 kPa and at a temperatureof 25° C. an unstable compound of MEA with CO₂, and the methane from the biogas not combined with MEA flows from chamber A of the saturator to the gas pipeline for methane 9a from where 34% of methane is forced to a gas main and 66% of methane flows to the gas mixer 11. In the gas mixer, cleaned biogas fed from tank 7 is enriched in methane, creating standard gas fuel. The solution of MEA with CO₂ flows from the low-temperature chamber A to the high-temperature of chamber B of the saturator under the same pressure 150 kPa. In chamber B of the saturator the solution undergoes thermal decomposition at a temperatureof 120° C. with the emission of gas, carbon dioxide and clean monoethyloamine. Carbon dioxide flows from chamber B to the gas pipeline for CO₂ 10d and monoethyloamine after being cooled to the temperature of 25° C. is returned by means of the liquid cycle of the saturator 8b to the low-temperature chamber A of the saturator. Further processing of CO₂, utilisation of standard gas fuel, and also generating electrical energy and thermal energy proceed as in example I.

1. The method of generating methane and electrical energy and thermal by means of an anaerobic conversion of biomass in the form of crushed plants and/or organic waste into biogas, and by employing a thermoregenerative cell and a current-generating unit or a current-generating turbo set to generate electrical and thermal energy, identifiable by the fact that crushed plants are mixed with water in such a proportion that the amount of dry mass in water is from 20% to 60%, preferably 30%; in the same proportion, crushed organic waste, initially containing less than 60% of water, is mixed with water, and these mixtures, together with organic waste containing from 4% to 20% of dry mass in water, undergo together, separately or in specific sets, hydrolysis at a temperatureof 20° C. over a period of 12-36 hours, then carbon dioxide is forced through the hydrolysed biomass until complete disappearance of oxygen and nitrogen in the biomass, then the biomass, alternatively completed with water until the amount of dry mass in water is from 4% to 60%, preferably 20%, undergoes methane fermentation by means of methane mesophile bacteria, preferably at a temperatureof 35° C., over the period of 48-240 hours, then the biogas produced in the process of an anaerobic conversion of biomass into biogas—further on called the first portion—is directed to a tank for raw biogas, whereas the rest of the biomass, alternatively completed with water until the amount of dry mass in water is from 4% to 60%, preferably 20%, undergoes methane fermentation by means of methane thermophile bacteria, preferably at a temperatureof 55° C., over the period of 48-240 hours, at the same time in both methane fermentation processes the proportion of carbon to nitrogen in the biomass is higher than 100:3, preferably 10:1, at pH 6-8 of the water mixture of the biomass, preferably pH=7 and its redox potential lower than 250 mV, then the biogas received in the anaerobic process of conversion of biomass into biogas by means of methane thermophile bacteria—further on called the second portion—is combined with the first portion in the tank for raw biogas and the rest of biomass, after subtracting of about 50% of water from it and returning this water to the methane fermentation process of the next portion of biomass, is composted, at the same time goes on the anaerobic process of converting biomass into biogas by means of methane psychrophile bacteria, preferably at a temperatureof 23° C. over the period of 190-300 hours, then the received compost is used in agriculture as natural fertiliser, and the created biogas, comprising the third portion, is combined with the first and second portions of the biogas; sulphur compounds are removed from them, and then 20%-80% of the desulphurised biogas is decomposed into methane and carbon dioxide, of which 5%-50% accumulates in a tank under higher pressure, and is then returned to the next process of removing of oxygen and nitrogen from the hydrolysed biomass, whereas the rest of the carbon dioxide is accumulated in gas bottles under higher pressure or is condensed or expelled to the atmosphere, whereas 25%-75% of methane is condensed, or combined with natural gas, or is used in its clean form as fuel, or is transformed into other chemical compounds, the rest of the methane or 100% of the received methane is combined with the desulphurised portion of biogas, which has not been decomposed, in the proportion necessary to get gas fuel of a constant methane number, preferably 104,4, and a constant heat value of 8,6 kWh/m³—called standard gas fuel, of which 20%-40% is burnt in the burner of a high-temperature thermoregenerator of a thermoregenerative cell and the substances of the thermal resolution of the synthesis products accumulated in the cell are returned from the high-temperature thermoregenerator to the electrodes of the cell generating electrical energy of direct current and synthesis products, whereas the rest of the fuel is burnt in the combustion engine of a current-generating unit generating electrical energy of variable current and heat contained in the liquids cooling the engine and in combustion gases, or is burnt in the combustion chamber of a current generating turbo set generating electrical energy of variable current and heat contained in the combustion gases emitted from the gas turbine, whereas 25%-75% of the heat regained from the liquids cooling the engine and from combustion gases is delivered to the low-temperature thermoregenerator of the thermoregenerative cell to the process of emitting of synthesis products from the electrolyte and returning them to the thermoregenerator of the high-temperature cell and returning to the cells of the cell of the electrolyte, which is characterised by lower concentration, whereas 25%-75% of heat is delivered to the processes of hydrolysis and anaerobic conversion of biomass into biogas, while the remaining heat is delivered to the heat cycle of central heating and/or for producing warm water.

2. The method according to claim 1, identifiable by the fact that the reflux created in each particular technological cycle is returned to be reused in the cycle.

3. The method according to claim 1, identifiable by the fact that reflux directed to the fermentation tanks is completed, in particular nitrogen compounds are added.

4. The system of generating methane and energy electrical and thermal composed of a hydrolyser, fermentation tanks, an expeller, a composter, a current generating unit or a current generating turbo set, a thermoregenerative cell, tanks, liquid and gas pumps and pipelines, identifiable by the fact that it contains a system of biomass preparation (1) connected to the hydrolyser (2), which in turn is connected to a series system of fermentation tanks and a composter (3) which is equipped with a conveyor of compost to a storage site and is connected to a system of returning and enriching reflux (4), moreover these systems: (1), (3) and (4) are connected to an outer water intake (15), a complex pipeline for raw biogas connects the series system of fermentation tanks and a composter (3) to a tank for raw biogas (5), in turn connected to a system for cleaning biogas (6), which in turn is connected to a tank for cleaned biogas (7), connected to a system of biogas decomposition (8) and a gas mixer (11), the system of biogas decomposition is connected to a system of carbon dioxide processing (10) and a system of methane processing (9); the system of carbon dioxide processing is connected by means of a gas pipeline to the hydrolyser (2) and it is also equipped with an outlet of CO₂ to the atmosphere, whereas the system of methane processing (9) is also connected to the gas mixer (11), which in turn is connected to a tank for standard gas fuel (12), this tank has a connection with a system of generating electrical energy and heat (13) and an alternative connection to a system of heat processing (14), whereas the system of electrical energy and heat generating (13) is connected to the system of heat processing (14), which in turn is connected by means of heat pipelines to the hydrolyzer (2), to the system of returning and enriching reflux (4) and the series system of fermentation tanks and a composter (3).

5. The system according to claim 4, identifiable by the fact that the system of biomass preparation (1) consists of a biomass mixer (1f) connected to the hydrolyser (2), and to the outer water intake (15) by means of a water pipeline of the biomass mixer (15a), it is also connected to a cutter (1d) of grass, leaves and cereal plants (1a) and to a cutter (1e) of root plants (1b); it is also connected to a storage site or a tank for organic waste (1c), especially when it is in the form of sedimentary solids in water.

6. The system according to claim 4, identifiable by the fact that the hydrolyser (2) is at the entrance connected to the system of biomass preparation (1); and at the exit it is equipped with a conveyor for hydrolysed biomass (2d), and a secondary water cycle of the hydrolyser (2a), coming out at the bottom of the hydrolyser from under the conveyor for hydrolysed biomass (2d), and getting in at the top of the hydrolyser near the entrance to the hydrolyser of biomass prepared by the system of biomass prepration (1), moreover the hydrolyser (2) is at the bottom equipped with a feeder of CO₂ to the hydrolyser (2b), and at the top there is an outlet (2c) of gases from the hydrolyser; it is also equipped with a water heater of the heating system of the hydrolyser and fermentation tanks (14c).

7. The system according to claim 4, identifiable by the fact that the series system of fermentation tanks and a composter (3) is composed of a mesophile fermentation tank (3a), a thermophile fermentation tank (3c), an expeller (3e) and a composter 3g, linked in series by means of biomass conveyors, at the same time the mesophile fermentation tank has at the entry a conveyor for hydrolysed biomass (2d), and at the outlet a conveyor for biomass after mesophile fermentation (3b); this conveyor is linked to the thermophile fermentation tank (3c), which at the outlet has a conveyor for biomass after thermophile fermentation (3d), connected to the expeller (3e), then the expeller is in turn connected by means of a conveyor for pressed biomass (3f) to the composter (3g), which is equipped with a leakproof gas chamber and at the outlet a conveyor of compost to the storage site (3h); both fermentation tanks are equipped with water heaters of the heating system of the hydrolyser and fermentation, tanks (14c), whereas the gas chambers of the fermentation tanks and composter are connected by means of gas pipelines to a tank for raw biogas (5,), connected by means of a pipeline for raw biogas (5a) to a system for cleaning biogas (6).

8. The system according to claim 4, identifiable by the fact that the system of returning and enriching reflux is composed of the secondary water cycle of the mesophile fermentation tank (4a), coming out at the bottom of the mesophile fermentation tank from under the conveyor for biomass after mesophile fermentation (3b) and getting into the fermentation tank at the top near the entry to the fermentation tank of the conveyor for hydrolysed biomass (2d), of the secondary water cycle of the thermophile fermentation tank (4c), getting out at the bottom of the thermophile fermentation tank (3c) from under the conveyor for biomass after thermophile fermentation (3d), and getting into the fermentation tank at the top near the entry into the fermentation tank of the conveyor for biomass after mesophile fermentation (3b), it is also composed of the secondary water intake of the expeller (4d) connected to the secondary water cycle of the thermophile fermentation tank (4c), and also of the secondary water cycle of the composter (4e), getting out at the bottom of the composter (3g), and getting into the composter at the top near the entry to the composter of the conveyor for pressed biomass (3f), both these cycles are connected to the outer water intake (15) by means of an outer water pipeline (15b), whereas the secondary water cycles of the mesophile fermentation tank (4a) and the thermophile fermentation tank (4c) are joined to a feeder of nitrogen compounds (4b).

9. The system according to claim 4, identifiable by the fact that the system of biogas decomposition (8b) consists of a two-chambered saturator (8a) and the liquid cycle of the saturator (8b), the entry chamber A of the saturator is filled with liquid absorbing only carbon dioxide and is equipped at the outlet with a gas pipeline for methane (9a), whereas inside the saturator, chamber A is linked to the exit chamber B of the saturator, filled with the same liquid emitting CO₂ and at the top connected to the gas pipeline for CO₂ (10d), and at the bottom to a pipeline for liquid of the liquid cycle of the saturator (8b), getting into chamber A and used for returning liquid from chamber B to chamber A, whereas chamber A of the saturator is connected by means of a gas pipeline below the liquid level in the chamber to a tank for cleaned biogas (7) and to a system of cleaning of raw biogas (6) consisting of a column for biogas desulphurisation (6a) and a gas pump (6b).

10. The system according to claim 4, identifiable by the fact that the system of carbon dioxide processing (10) is composed of a gas pipeline for carbon dioxide (10d) connecting the saturator (8a) and the feeder of CO₂ to the hydrolyser (2b), and moreover, a tank for compressed carbon dioxide (10c) and a condensing CO₂ unit (10a) are connected to the pipeline, the condensing CO₂ unit is connected from the other side to a tank for condensed carbon dioxide (10b), this pipeline also has a controlled outlet of carbon dioxide to the atmosphere (10e).

11. The system according to claim 4, identifiable by the fact that the system of methane processing (9) is composed of a gas pipeline for methane (9a) getting out of the saturator (8a) and connected to a methane condensing unit (9b), which is further on connected to a tank for condensed methane (9c) or connected to a gas main, also connected to a gas mixer (11), which is linked at the entrance to the tank for cleaned biogas (7), and at the exit to the tank for standard gas fuel (12).

12. The system according to claim 4, identifiable by the fact that the system of generating electrical energy and heat (13) is composed of a current-generating unit (13a), which has an electrical connection with a power network (13b) and a theromoregenerative cell (13c), which is equipped with a high-temperature thermoregenerator (13d) and a low-temprature thermoregenerator (13e), the combustion engine of the current-generating set and the high-temperature thermoregenerator of the cell are connected by means of a pipeline for standard gas fuel (12a) to the tank for standard gas fuel (12), and the pipeline (12a) has an anti-failure connection with a gas torch (12b), whereas the low-temperature thermoregenerator (13e) of the cell is also equipped with a heat exchanger connected to a heat exchanger combustion gases/liquid (14f) in the system of heat processing (14).

13. The system according to claim 4, identifiable by the fact that the system of heat processing (14) is composed of the main heat cycle of the hydrolyser and fermentation tanks (14c), the heat cycle of central heating (14d) and the heat cycle of the low-temperature thermoregenerator (14g), in the main heat cycle there is a water pump of the heat cycle (14a), connected by means of a heat pipeline (14b) a heat exchanger liquid/liquid (14e) in the cycle of liquids cooling engine, and then to a heat exchanger combustion gases/liquid (14f) absorbing heat from combustion gases, further on the main heat cycle is connected to a heat cycle of central heating (14d) and to the heating system of the hydrolyser and fermentation tanks (14c), equipped with water heaters situated in the hydrolyser and in the fermentation tanks, whereas the heat cycle of the low-temperature thermoregenerator (14g) connects the heat exchanger combustion gases/liquid (14f) to the heat exchanger of the low-temperature thermoregenerator (13e).

14. The system according to claim 12, identifiable by the fact that in the system of electrical energy and heat a gas turbine has been installed, which is connected at the synchro-tie to a three-faze current generator in place of the current-generating unit (13a), the pipeline for standard gas fuel (12a) is connected to the combustion chamber of the gas turbine, and the combustion outlet of the gas turbine is connected to a heat exchanger heating compressed air which is forced through to a gas fuel combustion chamber, and then in turn to a heat exchanger combustion gases/liquid (14f) in the main heat cycle of the system, whereas the three-faze current generator is connected electrically to a power network (13b).
